(19) United States
(12) Patent Application Publication
(10) Pub. No.: US 2020/0237715 A1
(43) Pub. Date: Jul. 30, 2020

(54) COX-2 Inhibitors for the Treatment of Ocular Disease

(71) Applicant: John James Vrbanac, JR., Portage, MI (US)

(72) Inventor: John James Vrbanac, JR., Portage, MI (US)

(21) Appl. No.: 16/840,346

(22) Filed: Apr. 4, 2020

Related U.S. Application Data
(60) Provisional application No. 62/919,913, filed on Apr. 5, 2019.

(51) Int. Cl. A61K 31/352; A61K 9/00

(52) U.S. Cl. CPC A61K 31/352; A61K 9/0019; A61K 9/0048

(57) ABSTRACT

There is provided a pharmaceutical composition suitable for topical administration to an eye, which contains a selective COX-2 inhibitory drug or salt thereof having a high water solubility and lipophilicity, concomitant with high potency, in a concentration effective for treatment and/or prophylaxis of a disorder in the eye, and one or more ophthalmically acceptable excipients that reduce rate of removal from the eye such that the composition has an effective residence time of about 1 to about 36 hours. Also provided is a method and kit for treating and/or preventing a disease or disorder in an eye, the method comprises administering to the eye a composition of the present disclosure.

### REFERENCE TO RELATED APPLICATIONS

This application claims priority to U.S. Provisional Application No. 62/919,913, filed Apr. 5, 2019, which is incorporated herein by reference.

### FIELD OF INVENTION

The present disclosure relates to pharmaceutical compositions containing a COX-2 inhibitor having defined hydrophobicity, water solubility, potency and/or resonance time parameters for topical administration to an eye for the treatment or prophylaxis of an ophthalmic disease or disorder in a subject in need thereof.

### BACKGROUND OF THE INVENTION

The estimated U.S. economic cost for impaired vision is a surprising $145 billion. Impaired vision effects 2.9 million Americans and 1.3 million are blind. Simply, blindness and low vision are economically and personally devastating. The total number of people in the U.S. affected by age-related macular degeneration (AMD) was 8,474,790 in 2013, and of these 361,600 were blind in both eyes, a number expected to rise to 501,600 by 2020 due to the aging population (41% increase in 7 years). Wet-AMD represents over half of the AMD market by cost of treatment. Current therapies for preventing or slowing the onset of blindness involve intravitreal (IVT) injections. Alternate treatment regimens are needed to improve current therapies.

Additionally, nonsteroidal anti-inflammatory drugs (NSAIDs) are used extensively in treating eye diseases and conditions. Indications include pain, photophobia, and photorefractive surgery. Other uses are to reduce inflammation and cystoid macular edema following cataract surgery. The US Food and Drug Administration has approved new topical NSAIDs and previously approved NSAIDs have been reformulated. For the most part, these represent “me too” products. One stated goal of new topical drugs and formulations are to allow for greater drug penetration into the retina, a goal that clearly has not been met. Some therapeutic effects on diabetic retinopathy and age-related macular degeneration with IVT have been described, but no drugs have been approved for topical administration to treat RDD, representing a stated unmet medical need. Topical diclofenac (0.1%) and bromfenac (Sun Pharmaceutical Industries, Inc., USA) (0.09%) have been studied in wet-AMD, always in combination with another therapy such as photodynamic therapy (PDT), intravitreal (IVT) ranibizumab (LUCENTIS®, Genentech, Inc., USA), or IVT bevacizumab (AVASTIN®, Genentech, Inc., USA). The results have been mixed with no benefit or a statement of “may add benefit,” etc. This being noted, the results with bromfenac were more consistently positive than diclofenac (Glaxo-SmithKline, USA). In summary, there is strong interest in a topical NSAID that reaches the retina in active concentrations over a long period of time for standalone or combination therapy. A need is clearly expressed in the clinical literature.

Interest has been expressed in the literature to determine what physico-chemical properties are necessary for efficient delivery of drug from the surface of the eye to the retina or retina-choroid. Certain drugs are clearly delivered to the retina following topical instillation in pharmacologically active concentration, timolol (Akorn, Inc., USA) and brimonidine representing two of the most studied drugs.

RDDs represent unmet therapeutic needs where the patient population is being underserved, effecting millions worldwide and in the US. For preservation of vision, patients with wet-AMD often require monthly injections into the eye. Current therapies involve the injection of a large molecule that tightly binds vascular endothelial growth factor (VEGF) and include antibodies (AVASTIN®) and antibody fragments (LUCENTIS®). Other agents are fusion proteins such as EYLEA® (Regeneron Pharmaceuticals, Inc., USA) (VEGF binding portion from the VEGF-receptor bound to the Fc portion of the human IgG1 immunoglobulin) and aptimers (MACUGEN®, NeXstar Pharmaceuticals, Inc., USA). Most notably, these treatments typically work for 2-3 months before another IVT injection in the ophthalmologists office is required, a less than optimal treatment regimen.

Further, the vast majority of the drugs currently under development target neovascularization via VEGF. The Port Delivery System or PDS is implanted into the eye wall and slowly releases the anti-VEGF drug Lucentis® (Phase II). Brolucizumab (Novartis AG, Switzerland) is a newer anti-VEGF drug and is a humanized single-chain antibody fragment under development by more than one company and may be approved in 2019. The anti-VEGF binder abicipar (Molecular Partners AG, Switzerland) is a so-called DARPin®, genetically engineered antibody mimetic proteins, which often exhibit both highly specific and high-affinity binding. RGX-314 (RegenXBio, USA) is a gene therapy (anti-VEGF gene). Antibody therapies that target two different proteins, angiopoietin II and VEGF, are RG7716 (Roche, Switzerland) and REGN 901-3 (Regeneron Pharmaceuticals, Inc., USA), a highly promising approach in theory offering the possibility of combination-drug synergy. A small-molecule administer by IVT injection that targets the VEGF receptor is sunitinib (Pfizer Inc., USA) (multi-targeted receptor tyrosine kinase (RTK) antagonist originally target for cancer). Other drugs include OPT-302 (Opthea Limited, USA) and a dorzolamide-timolol combination. Additional drug targets under examination are the proteins endoglin, activin and tissue factor which are involved in angiogenesis, a central phenomenon in wet-AMD.

Drugs co-injected with anti-VEGF drugs and targeting complement factors are APL-3 (Apellis Pharmaceuticals, Inc., USA) and ZIMURA® (avacincaptad pegol, Ophthotech Corporation, USA). Again, this approach is theoretically attractive since drug combinations with different mechanisms of action offer the real possibility of drug synergy, instead of an additive drug-combination effect.

PAN-90806 (PanOptica, Inc., USA) is a drug with pharmacology virtually identical to sunitinib and is currently in Phase II/III Clinical Trials for the treatment of neovascular age-related macular degeneration.

Therefore, these and other unmet needs will be fulfill by the current disclosure.

### SUMMARY OF THE INVENTION

In one embodiment, a pharmaceutical composition suitable for topical administration to an eye is provided, the composition comprises a selective cyclooxygenase-2 (COX-2) inhibitory drug or a prodrug or salt thereof, for the treatment or prophylaxis of an ophthalmic disease or disorder. In another embodiment, the composition is in a therapeutically-effective concentration useful for the treatment and/or prophylaxis of wet age-related macular degeneration disease in the eye. The composition may include at least one ophthalmically acceptable excipient. In still another embodiment, the selective COX-2 inhibitory drug exhibits a Log P (hydrophobicity) in a range between a Log P of about 2.5 to about 5.5, or equal to or greater than a Log P of about 2.5, or equal to or greater than a Log P of about 5.1, or equal to or greater than about 5.5. In another embodiment, the selective COX-2 inhibitory drug exhibits a water solubility (hydrophilicity) of equal to or greater than about 0.2 mg/ml, or equal to or greater than about 40 mg/ml, or equal to or greater than about 20 mg/ml, or equal to or greater than about 5 mg/ml, or equal to or greater than about 1 mg/ml. In yet another embodiment, the selective COX-2 inhibitory drug exhibits a potency (enabling mean resonance time above Ki) of less than or equal to about 50 nM, or less than or equal to about 25 nM, or less than or equal to about 10 nM, or less than or equal to about 5 nM, or less than or equal to about 1 nM. In still another embodiment, the resonance time of the selective COX-2 inhibitory drug in the eye is equal to or greater than about 60 minutes, or equal to or greater than about 120 minutes, or equal to or greater than about 180 minutes, but less than or equal to about 36 hours, or less than or equal to about 24 hours, or less than or equal to about 18 hours, or less than or equal to about 12 hours, or less than or equal to about 8 hours. In another embodiment, the pharmaceutical composition is applied external to the eye using a topical drop formulation. In one embodiment, a topical application of a COX-2 inhibitory drug is used in combination therapy for the treatment or prophylaxis of wet-age-related macular degeneration.

In yet another embodiment, a method of treating a cyclooxygenase-2 mediated ophthalmic disease or disorder in a subject is provided. The method comprises treating a subject having or is susceptible to an ophthalmic disease or disorder with a therapeutically-effective amount of a selective cyclooxygenase-2 inhibitory drug having a Log P between about 2.5 to about 5.5. In yet another embodiment, the ophthalmic disease or disorder is wet age-related macular degeneration.

In another embodiment, a kit is provided that contains one or more of the following: a) at least one selective cyclooxygenase-2 inhibitory drug or a pharmaceutically acceptable prodrug or salt thereof, b) at least one ophthalmically acceptable ingredient, and 3) a device for administering the at least one selective cyclooxygenase-2 inhibitory and/or the at least one ophthalmically acceptable ingredient to an eye of a subject. In yet another embodiment, the kit is used to administer at least one selective cyclooxygenase-2 inhibitory drug to a subject that has or is susceptible to a wet age-related macular degeneration disease.

Other features and advantages of the present disclosure will be in part apparent and in part pointed out hereinafter.

### DESCRIPTION OF ILLUSTRATIVE EMBODIMENTS

IVT injections are uncomfortable and invasive whereas topical drug instillations are comfortable procedures and non-invasive. This advantage is clear from the patient's perspective. Another advantage to a topical COX-2 inhibitor active in the retina for hours in that this will allow the ophthalmologist to use combination therapy at their discretion, a therapeutic advantage in real-life ophthalmology office situations. This approach is attractive since drug combinations with different mechanisms of action offer a real possibility of drug synergy instead of an additive drug-combination effect.

Conventional wisdom dating back 20 years was that it was not possible to deliver drugs topically to the retina or retina-choroid topically in therapeutic amounts, at least for a resonance time that would remain active in term of hours instead of minutes. Hence, there are currently no topical ocular drug treatments for retinal degenerative diseases (RDDs), including wet-AMD, although a vascular endothelial growth factor (VEGF) receptor small molecule inhibitor, PAN-90806 (PanOptica, Inc., USA), is currently in Phase II/III clinical trials and may become available in the next 1-2 years. Therefore, there is a clear need for an effective, topically active inhibitor for the treatment of wet-AMD and other neovascular eye diseases, which is applied to the surface of the eye and delivered to the retina in efficacious concentration-time parameters sufficient to treat eye disease.

Further, inflammation is involved to differing extents in most diseases. The formation of new blood vessels—neovascularization—is a phenomenon that is central to the morbidity and mortality of certain diseases, including specific retinal degenerative diseases (RDDs). These two phenomenon, inflammation and neovascularization, interact in highly complex ways and simultaneous drug treatment for both may represent a therapeutic goal in different clinical situations. It is generally appreciated that increases in cell proliferation, invasion and angiogenesis are important to varying extents in RDDs and other diseases such as cancers.

It is also appreciated that drug distribution characteristics such as oral bioavailability and distribution to the central nervous system require both some water solubility and some lipophilicity, as understood by those skilled in the art. With respect to the sojourn from the surface of the eye to the retina or retina-choroid, the combination of both of these two physico-chemical properties is important, but not well understood. Provided that other phenomenon such as transporters are not involved, lipophilicity is important in passage through the plasma lemma of cells and through intracellular membranes (mitochondria, for example). This is widely appreciated. However, the critical path from the surface of eye to the back of the eye which involves movement through the sclera is not appreciated, as is the role of “high” water solubility in this transport. The sclera is mostly connective tissue with a sparse population of cells and drugs must dissolve in this aqueous medium and then move—by simple diffusion through an aqueous medium—to the back of the eye.

Although not wishing to be bound by theory, it is believed the following non-exclusive factors affect the delivery of a compound or drug to the retina from the surface of the eye to achieve a resultant pharmacologically significant effect: 1) surface resonance time; 2) flux (permeability) through the barriers of conjunctiva, sclera and RPE; 3) the role of blood and lymph flow to remove drug; 4) tissue binding in target tissues; 5) Ki at the drug target; and/or 6) resonance time on the surface of the retina related to dosing (for example, single dosing vs. multiple dosing) and/or other factors such as gelling agents, hydrogels, and other drug delivery agents. It is widely believed that factors 1 and 6 are generally the same or very similar phenomenon. Factors 3 and 4 are considerations that are typically not under a drug scientists control (unless melanin binding, for example, was specifically included in early screens as necessary properties). However, it has not been recognized until now that for a selective COX-2 inhibitor to be useful for ocular drug delivery from the surface of the eye to the retina, that a combination of Factor 2 and Factor 5 having high water solubility, high hydrophobicity, and low nM potency are important to achieve the desired pharmacologically significant effect described in the present disclosure.

Thus, when examining the physico-chemical properties of a potential drug candidate for ocular drug delivery from the eye surface to retina, properties such as high water solubility, high hydrophobicity, and/or low nM potency are important factors when considering drug candidates useful in the present disclosure.

In an illustrative embodiment, a COX-2 inhibitor, such as the COX-2 inhibitor 6.8-Dichloro-2-trifluoromethyl-2H-1-benzopyran-3-carboxylic acid, and/or related compounds, prodrugs or salts thereof, having physico-chemical properties capable of crossing the sclera of the eye following drug administration outside of the eye, can be used in the present disclosure either with a or with topical instillation (drops) or periocular DEPOT injection. In one embodiment, a subconjunctival DEPOT injection of the ethyl ester of a prodrug of a COX-2 inhibitor, such as SD-8381 (described herein), formulated in sesame oil is delivered across the sclera resulting in μM concentrations of drug in both the retina and in the choroid to achieve the therapeutic effect in a subject in need thereof.

As used herein the phrase “high water solubility” is defined as water solubility of equal to or greater than about 40 mg/ml, or equal to or greater than about 30 mg/ml, or equal to or greater than about 20 mg/ml, or equal to or greater than about 10 mg/ml, or equal to or greater than about 5 mg/ml, or equal to or greater than about 2 mg/ml, or equal to or greater than about 1 mg/ml, or equal to or greater than about 0.5 mg/ml, or equal to or greater than about 0.4 mg/ml, or equal to or greater than about 0.3 mg/ml, or equal to or greater than about 0.2 mg/ml, or equal to or greater than about 0.1 mg/ml, or equal to or greater than about 0.05 mg/ml, or equal to or greater than about 0.02 mg/ml.

As used herein, the phrase “high hydrophobicity” is defined as hydrophobicity in the range of a Log P between of about 2.5 to about 5.5, or equal to or greater than a Log P of about 2.5, or equal to or greater than a Log P of about 5.1, or equal to or greater than about 5.5.

As used herein, the phrase “high potency” is defined as a potency (enabling mean resonance time above Ki) of less than or equal to about 50 nM, or less than or equal to about 25 nM, or less than or equal to about 10 nM, or less than or equal to about 5 nM, or less than or equal to about 1 nM.

As used herein, the phrase “residency time” or “resonance time” is defined as the time in the eye equal to or greater than about 60 minutes, or equal to or greater than about 120 minutes, or equal to or greater than about 180 minutes, but less than or equal to about 36 hours, or less than or equal to about 24 hours, or less than or equal to about 18 hours, or less than or equal to about 12 hours, or less than or equal to about 8 hours.

A selective COX-2 inhibitory compound that may be useful herein can be any such drug known in the art, including, without limitation, compounds disclosed in the patents and publications listed below, which possess the hydrophobicity, water solubility, potency and/or residency time in the eye described herein:
U.S. Pat. No. 5,466,823 to Carter et al.,
U.S. Pat. No. 5,972,986 to Seibert et al.,
U.S. Pat. No. 6,034,256 to Carter et al.,
U.S. Pat. No. 6,822,102, to Rogier, Jr., et al.,
U.S. Pat. No. 8,846,744 to Ranzani et al.
U.S. Pat. No. 8,580,827 Chen et al.,
U.S. Pat. No. 8,410,287 to Remenar et al.,
U.S. Pat. No. 8,778,400 to Prestidge et al.,
U.S. Pat. No. 9,061,004 to Markowitz et al.,
U.S. Pat. No. 9,770,432 to Zhang et al.,
U.S. Patent Publication No. 20090298797 to Zheng et al.,
U.S. Patent Publication No. 20100233272 to Appel et al.,
U.S. Patent Publication No. 20130203781 to Pridgen,
U.S. Patent Publication No. 20130203783 to Pridgen,
U.S. Patent Publication No. 20130072534 to Ces et al., and
U.S. Patent Publication No. 20130028937 to Salaman et al.
   For example, a useful COX-2 inhibitory drug disclosed in U.S. Pat. No. 6,034,256 is:
6.8-Dichloro-2-trifluoromethyl-2H-1-benzopyran-3-carboxylicacid.
   Another example of COX-2 inhibitory drugs useful in the present disclosure disclosed in U.S. Pat. No. 6,822,102, is for example:
(2S,3R)-6,8-dichloro-3,4-dihydro-2-(trifluoromethyl)-2H 1-benzopyran-3-carboxylicacid.

Other selective COX-2 inhibitory drugs useful herein include those described in International Patent Publication No. WO 98/47890 to Jeffery S. Carter et al., which discloses benzopyran COX-2 inhibitors. Although there is a general disclosure that the compounds may be useful in the treatment of post-ophthalmic surgery inflammation, such as cataract surgery and refractive surgery, and in the treatment of ophthalmic diseases, such as retinitis, retinopathies, conjunctivitis, uveitis, ocular photophobia, acute injury to the eye tissue, glaucoma and sarcoidosis, or used in the treatment of ophthalmological conditions such as corneal graft rejection, ocular neovascularization, retinal neovascularization including neovascularization following injury or infection, diabetic retinopathy, macular degeneration, retrolental fibroplasia and neovascular glaucoma, there is no suggestion or disclosure that the benzopyran COX-2 inhibitors disclosed therein possess the high hydrophobicity, high water solubility, high potency and/or resonance time (time when drug concentration remains greater than the Ki) parameters disclosed in the current disclosure, which would allow such compounds to be useful in topical administrations to the eye for the treatment or prophylaxis of ophthalmic diseases or disorders described herein.

Still other selective COX-2 inhibitory drugs useful herein that possess the hydrophobicity, water solubility, potency and residency time in the eye described herein are disclosed in, for example:
U.S. Pat. No. 5,466,823 to Talley et. al.,
U.S. Pat. No. 5,633,272 to Talley et al.,
U.S. Patent Publication No. 20050010050 to Rogier et al.,
U.S. Patent Publication No. 20050148627 to Cater et al.,
International Patent Publication No. WO 13/189121,
International Patent Publication No. WO 09/500501,
International Patent Publication No. WO 02/083655,
International Patent Publication No. WO 04/010945,
International Patent Publication No. WO 04/002420,
International Patent Publication No. WO 04/002409,
International Patent Publication No. WO 04/037798,
International Patent Publication No. WO 03/055874,
International Patent Publication No. WO 03/055875,
International Patent Publication No. WO 04/048347,
International Patent Publication No. WO 04/014878,
International Patent Publication No. WO 04/046121,
International Patent Publication No. WO 03/031409,
International Patent Publication No. WO 06/099416,
International Patent Publication No. WO 03/029217,
International Patent Publication No. WO 07/031829,
International Patent Publication No. WO 03/031418,
International Patent Publication No. WO 06/040676,
International Patent Publication No. WO 03/031398,
International Patent Publication No. WO 05/051941,
International Patent Publication No. WO 05/068442,
International Patent Publication No. WO 05/007620,
International Patent Publication No. WO 00/042021,
International Patent Publication No. WO 03/091221,
International Patent Publication No. WO 03/090730,
International Patent Publication No. WO 05/014546,
International Patent Publication No. WO 06/079923,
International Patent Publication No. WO 03/078408,
International Patent Publication No. WO 03/041705, and
International Patent Publication No. WO 05/065684.

One advantage COX-2 inhibitors have over conventional NSAIDs for application to the eye is the lack of effect on baseline COX-1 mediated physiological functions, including wound healing following eye surgery and intraocular pressure control. Further, therapeutic and prophylactic methods involving NSAIDs generally lack selectivity for inhibition of COX-2, thereby increasing the risk of side-effects commonly associated with COX-1 inhibition. Therefore, highly effective relief or prevention of COX-2 mediated ophthalmic disorders can be obtained with greatly reduced risk of the side-effects commonly associated with COX-1 inhibition, when using a selective COX-2 inhibitor as compared to an NSAID. Thus the method of the present disclosure is particularly suitable where conventional NSAIDs are contraindicated, for example in subjects with peptic ulcers, gastritis, regional enteritis, ulcerative colitis or diverticulitis, subjects with a recurrent history of gastrointestinal lesions, patients with gastrointestinal bleeding, coagulation disorders including anemia such as hypothrombinemia, hemophilia and other bleeding problems, or kidney disease, patients prior to surgery, or subjects taking anticoagulants.

The term “ophthalmically acceptable” as used herein with respect to a formulation, composition or ingredient of the present disclosure means having no persistent detrimental effect on the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, exemplary formulations, compositions and ingredients are those that cause no substantial detrimental effect, even of a transient nature.

The term “prevention” or “prophylaxis” includes either preventing the onset of clinically evident ophthalmic disease altogether or preventing the onset of a preclinically evident stage of an ophthalmic disease or disorder in individuals. This includes prophylactic treatment of those at risk of developing a disease, such as wet-AMD, for example.

The phrase “therapeutically-effective” is intended to qualify the amount of each agent that will achieve the goal of improvement in disease or disorder severity, longevity and/or the frequency, while avoiding adverse side effects typically associated with alternative therapies.

Ophthalmically acceptable ingredients may be added to a formulation of the present disclosure to assist in delivery of the active inhibitory agents to the eye. Examples of ophthalmically acceptable ingredients are well known in the art, for example, those described in US 20000128267A1, to R. Bandyopadhyay, et al.

Also included in compounds described herein are the stereoisomers thereof. Compounds of the present disclosure can possess one or more asymmetric carbon atoms and are thus capable of existing in the form of optical isomers as well as in the form of racemic or nonracemic mixtures thereof. Accordingly, some of the compounds of this disclosure may be present in racemic mixtures which are also included in this disclosure. The optical isomers can be obtained by resolution of the racemic mixtures according to conventional processes, for example by formation of diastereoisomeric salts by treatment with an optically active base and then separation of the mixture of diastereoisomers by crystallization, followed by liberation of the optically active bases from these salts. Examples of appropriate bases are brucine, strychnine, dehydroabietylamine, quinine, cinchonidine, ephedrine, α-methylbenzylamine, amphetamine, deoxyphedrine, chloramphenicol intermediate, 2-amino-1-butanol, and 1-(1-napthyl)ethylamine. A different process for separation of optical isomers involves the use of a chiral chromatography column optimally chosen to maximize the separation of the enantiomers. Still another available method involves synthesis of covalent diastereoisomeric molecules. The synthesized diastereoisomers can be separated by conventional means such as chromatography, distillation, crystallization or sublimation, and then hydrolyzed to deliver the enantiomerically pure compound. The optically active compounds can likewise be obtained by utilizing optically active starting materials. These isomers may be in the form of a free acid, a free base, an ester or a salt. Additional methods for resolving optical isomers, known to those skilled in the art may be used, for example, those discussed by J. Jaques et al in Enantiomers, Racemates, and Resolutions, John Wiley and Sons, New York (1981).

Also included in the compounds described herein are the amide protected acids thereof. Thus primary and secondary amines can be reacted with the chromene-3-carboxylic acids to form amides which can be useful as prodrugs. Illustrative amines heterocyclicamines, include optionally substituted aminothiazoles, optionally substituted amino-isoxazoles, and optionally substituted aminopyridines; aniline derivatives; sulfonamides; aminocarboxylic acids; and the like. Additionally, 1-acyldihydroquinolines can behave as prodrugs for the 1H-dihydroquinolines.

Other amides prodrugs useful in the present disclosure are disclosed in Qandil, Int. J. Mol. Sci. 2012, 13, 17244-17274, which is incorporated by reference herein, and in U.S. Pat. No. 7,420,061 to Talley et al., and U.S. Pat. No. 6,809,111 to Carter et al.

Also included in the compounds useful in the present disclosure are the pharmaceutically-acceptable salts thereof. The term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds useful in the present disclosure may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicyclic, salicyclic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, β-hydroxybutyric, salicyclic, galactaric and galacturonic acid. Suitable pharmaceutically-acceptable base addition salts of compounds useful in the present disclosure include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the present disclosure by reacting, for example, the appropriate acid or base with the desired compound.

Any drug having utility as a topical ophthalmic application can be used in co-therapy, co-administration or co-formulation with a composition or formulation of the present disclosure. Such drugs include, for example, demulcents; antibiotics, antivirals and other anti-infectives; steroids, NSAIDs and other anti-inflammatory agents; acetylcholine blocking agents; antiglaucoma agents including beta-adrenergic receptor blocking agents, carbonic anhydrase inhibitors and prostaglandins; antihypertensives; antihistamines; anticataract agents; and topical and regional anesthetics. Illustrative specific drugs include acebutolol, aceclidine, acetylsalicylic acid (aspirin), N⁴ acetylsulfisoxazole, alclofenac, alprenolol, amfenac, amiloride, aminocaproic acid, p-aminoclonidine, aminozolamide, anisindione, apafant, atenolol, bacitracin, benoxaprofen, benoxinate, benzofenac, bepafant, betamethasone, betaxolol, bethanechol, bimatoprost brimonidine, bromfenac, bromhexine, bucloxic acid, bupivacaine, butibufen, carbachol, carprofen, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, interferons, isobutylmethylxanthine, isofluorophate, isoproterenol, isoxepac, ketoprofen, ketorolac, labetolol, lactorolac, latanoprost, levo-bunolol, lidocaine, linezolid, lonazolac, loteprednol, meclofenamate, medrysone, mefenamic acid, mepivacaine, metaproterenol, methanamine, methylprednisolone, metiazinic, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, travoprost, triamcinolone, trimethoprim, trospectomycin, isopropyl unoprostone, vancomycin, vidarabine, vitamin A, warfarin, zomepirac and pharmaceutically acceptable prodrugs and salts thereof.

Besides being useful for human treatment, these compounds, methods and formulations are also useful for veterinary treatment of companion animals, exotic animals and farm animals, including, for example, mammals, rodents, and the like, such horses, monkey, dogs, rat, rabbit, and cats.

Illustratively, to demonstrate efficacy in vivo, a predictive animal model in a rabbit is used. For example, the efficacy of the COX-2 inhibitor 6.8-Dichloro-2-trifluoromethyl-2H-1-benzopyran-3-carboxylicacid delivered via intravitreal (IVT) injections to the site of action for the treatment of wet-AMD, regardless of the route-of-administration (ROI), the extent and duration of inhibitory actions of the COX-2 inhibitor on chronic retinal neovascularization (RNV) induced by intravitreal (IVT) injection of DL-α-aminoadipic acid (AAA) in a rabbit model using fluorescein angiography is used. Intravitreal (IVT) injections deliver this compound to the site of action and these results are predictive. Topical and subconjunctival DEPOT injection subsequent to IVT are expected to be effective for this specific molecule.

Other compounds with properties of a relatively high water solubility (for example, about 3-4 mg/mL, or greater), a relatively high lipophilicity (for example, about a Log P of about 3, or greater) and/or, a high affinity, Michaelis-Menten binding kinetics, for the target receptor (for example, a Ki of less than about 10 nM), will be delivered to the retina in preclinical species such as rabbit, dog and monkey in pharmacologically active concentrations. Other combinations of water solubility, lipophilicity and/or Ki will also be evaluated. In addition the compounds will be tested for the presence in the eye above the Ki for a period of time (mean resonance time) that is measure in term of hours. The specific compound, the involvement of other distribution phenomena such as transporters, the species, the specific protein target and the specific tissue elimination kinetics will vary the observed pharmacokinetic and pharmacodynamic responses.

To demonstrate the in vivo efficacy of a COX-2 inhibitor useful in the present disclosure that is delivered external to the eye, such as, for example, via topical drops, suppression of leakage can be first shown by establish efficacy via intravitreal (IVT) injection of a subject. Once IVT efficacy is established, other external routes of administration can be examined. For example, chronic retinal neovascularization (RNV) and leakage has been established with IVT injection of DL-α-aminoadipic acid (DL-AAA) which is currently a recognized predictive animal model of wet-AMD in rabbit. The scientific literature indicates that COX-2 inhibitors can result in drug-induced attenuation of macrophage infiltration and down-regulation of VEGF in models of RNV. Topical and/or subconjunctival delivery can be examined subsequent to IVT.

Although this invention has been described with respect to specific embodiments, the details of these embodiments are not to be construed as limitations.

In regard to dosages, individual needs vary, but determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The amount of a given COX-2 selective inhibitor of the invention that will be effective in the treatment of a particular disorder or condition will depend on the nature of the disorder or condition, and can be determined by standard clinical techniques, including reference to Goodman and Gilman, supra; The Physician's Desk Reference, Medical Economics Company, Inc., Oradell, N J., 1995; and Drug Facts and Comparisons, Inc., St. Louis, Mo., 1993. The precise dose to be used in the formulation will also depend on the route of administration, and the seriousness of the disease or disorder, and should be decided by the physician and the patient's circumstances.

The compounds may be administered on a regimen of up to 6 times per day, preferably 1 to 4 times per day, and most preferably once per day. Effective doses may be extrapolated from dose-response curves derived from in vitro or animal model test systems and are in the same ranges or less than as described for the commercially available compounds in the Physician's Desk Reference, supra.

The invention also provides pharmaceutical kits comprising one or more containers filled with one or more of the ingredients of the pharmaceutical compounds and/or compositions of the disclosure, including, at least, one or more of the COX-2 selective inhibitors. Associated with such kits can be additional therapeutic agents or compositions, devices for administering the compositions, and notices in the form prescribed by a governmental agency regulating the manufacture, use or sale of pharmaceuticals or biological products which reflects approval by the agency of manufacture, use or sale for humans.

The present disclosure, while not limited thereto, will be further illustrated by the following examples.

### EXAMPLES

The following examples are included to demonstrate particular embodiments of the invention. It should be appreciated by those of skill in the art that the techniques disclosed in the examples which follow represent techniques that may be modified or altered and are not intended to limit the scope of the present disclosure.

### Example 1

### Phase I Hypothesis/Rationale

Inhibition of the COX-2 receptor has been demonstrated to reduce cystoid macular edema in patients following ocular surgery and suppress neovascularization and reduce edema in animal models of wet AMD and diabetes. Test compounds having initial properties of a water solubility of 20 mg/mL, a Log P of 5.1 and a Ki for the COX-2 enzyme of 5 nM will first be tested in a preclinical rabbit model for wet-AMD. Based upon our observations that compounds that have the unusual properties of the combination of high water solubility and high lipophilicity, it is believed that the compound will be delivered to the retina and retina-choroid following topical administration. In addition, it is believed highly potent drugs (for example, a Ki of <10 nM) will be present in the retina or retina/choroid above the Ki for a period of time (mean resonance time) that is measured in hours instead of minutes. It is appreciated that the specific compound evaluated, the involvement of other distribution phenomena such as transporters, the species used, the specific protein targeted and tissue elimination kinetics will play a role in the observed pharmacodynamic and pharmacokinetic response observed. Further, other compounds with other combinations of water solubility, Log P and Ki for the COX-2 enzyme will also be tested based on the results from this initial study.

Development of drugs to treat the retina via topical or peri-ocular routes of administration is ideally done in a large eyed animal, such as a rabbit, dog or primate. Therefore, a rabbit model of chronic retinal vascular leakage which mimics several aspects of the human condition will be used.

### Example 2

### Phase II Hypothesis

Phase II research will examine 2-3 additional COX-2 inhibitors in this chemical space as defined and superiors efficacious compounds will be chosen to further the research studies. Additional models will be employed including the dog and the monkey AAA model. Further formulations and ocular PK work will be completed. Ocular GLP safety assessment will also be completed.

### Example 3

### Phase I Specific Aims

It is anticipated that the delivery of test compound external to the eye via topical drops or subconjunctival depot injection will be well tolerated and reduce chronic leakage established in the DL-AAA model in rabbit.

### Example 4

### Rabbit DL-AAA Model of Wet AMD

Intravitreal injection of the glial toxin DL-2-aminoadipic acid (DL-AAA) has been shown to cause neovascularization and retinal leakage in NZW rabbit, DB Rabbit, RCS rat, and African Green Monkey. (Burke, -Hu). Following injection of DL-AAA, neo-vascularization with permeable vessels occurs over the first 6-8 weeks. These new leaky vessels remain permeable for up to 24 months without treatment. Treatment with IVT delivered anti-VEGF therapies such as Avastin or Rikki's have been shown to suppress leakage for a period of time, however as drug is cleared from the eye leakage returns. This is a significant advantage over current models, in that it is possible to not only evaluate the potency of a drug to suppress leakage, but also the duration of action. This is quite similar to the chronic nature of human retinal vascular diseases, such as Wet-AMD and DME, where the disease is in progress before treatment begins, and as a treatment is cleared from the system, the pathology returns. Negative controls are not necessary in the DL-AAA model since both pre-treatment leakage and terminal leakage serve as baseline pathology.

This experiment will include up to three parts: Part 1, test article tolerability, Part 2, evaluation of topical dosing schedules, and Part 3, evaluation of subconjunctival dosing. If topical delivery of the test article sufficiently suppresses leakage, Part 3 may not be performed. It is also important to note that with the DL-AAA model leakage returns naturally without the need for additional induction article. Therefore, the same animals can be used to evaluate multiple treatment paradigms. This greatly reduces the cost of development and need for additional animals. There is no concern for animal welfare since all treatments will be screened for tolerability in naive animals first.

In Part 1, the tolerability of the test article in naive DB rabbits (2M, 2F) will be evaluated. Test compounds will be for topical formulations. Typical ocular topical formulations to be considered including PBS and gelling/dissolution agents such as hydroxypropyl-methylcellulose. Animals will begin receiving the lowest concentration of test article 1× per day for 3 days. Ophthalmic exams will be performed by a veterinary ophthalmologist daily to look for signs of irritation and discomfort. Dosing frequency and drug concentration will be increased up to 3× per day and a 0.5% (w/v) suspension in 1.0% hydroxypropyl-methylcellulose (w/v) or until a non-tolerable dosing schedule is reached. If sufficient efficacy is not established in Part 2 via topical delivery of TA, additional animals will be used (2M, 2F) to assure the tolerability of a subconjunctival route of administration. Following subconjunctival injections, animals will be examined for at least 2 weeks. In addition to ophthalmic exams, fundus photography and OCT will be performed and eyes will be harvested and processed for histopathology.

In Part 2, the reduction of leakage provided by TA in the DL-AAA model will be measured. DB rabbits (4M, 4F) will be injected with DL-AAA on Day 0. On Week 10, fluorescein angiography (FA) will be performed to measure baseline leakage. Animals will initially be dosed using the highest tolerated frequency and drug concentration determined from Part 1. Changes in retinal leakage will be measured over the following 2 weeks. If leakage is suppressed sufficiently, a 2 week wash out period will be allowed to reestablish leakage and a lower concentration and or frequency of dosing will be evaluated in the same animals. If leakage is not suppressed sufficiently using the highest tolerated frequency and drug concentration Part 3 will be required.

In Part 3, the animals for Part 2 will be allowed a +2-week wash out period while an additional tolerability study is conducted (see Part 1). Test formulations will be injected on Day 0 changes in retinal leakage will be measured every Day 2, 5, 7 and bi-weekly there after until leakage returns to baseline or 3 months have passed.

Due to the properties of the test formulations, it is expected that a statistically significant reduction in retinal leakage, with a tolerated topical dose, will occur. Alternatively, if topical dosing does not deliver drug at sufficient concentrations to suppress retinal leak, it is anticipated that the higher retinal concentrations attainable with subconjunctival dosing will reduce leakage in this model.

It should be noted that formulation of a test compound and route of administration greatly effect target tissue bioavailability. If neither topical or subconjunctival delivery is efficacious additional formulations may need to be evaluated. Additionally, it is possible that COX-2 inhibition may not be sufficient to reduce established chronic leakage. In this case other disease models may be investigated.

We further propose to determine the concentration of drug in ocular tissues and further evaluate non-GLP safety and tolerability following an efficacious dosing schedule established in SA1. Assuming that an efficacious schedule was established, the pharmacodynamic/pharmacokinetic relationship will be established as with all pharmacology studies. If an efficacious dosing schedule was not established in SA1, PK will be performed using the highest tolerated dose in order to determine if drug is getting to the target tissues and the safety evaluation (Part 3) of this SA will not be conducted.

A discovery level of validation, non-GLP quantitative method for test compound detection in ocular tissues (conjunctiva, sclera, choroid and retina) will be developed using a modern Q-Tof or triple quadrupole LC-MS instrument.

Terminal and C max PK will be determined for topical administration and terminal PK for subconjunctival administration. For topical administration, one time point will be just prior to dosing and after 3 days of dosing and C max will be 10 minutes after dosing (after 3 days of dosing). For the conjunctival administration, samples will be taken on day-3. Satellite groups will be used. Two animals will be utilized for each dose and time point. Samples taken and analyzed will be conjunctiva, anterior sclera, posterior sclera, choroid and retina. Additional samples may be taken and frozen. Posterior sclera, choroid and retina samples will be taken near the fovea. Three doses will be used for topical: Maximally effective dose, NOEL dose and ⅓ maximal effective dose. One dose will be examined for conjunctiva DEPOT. Topical samples=5 tissues, 2 time points, two animals per time point=4 eyes, 3 doses=120 samples. Conjunctiva DEPOT=5 tissues, 1 time point, two animals per time point=4 eyes, 1 doses=20 samples. A satellite group of animals will be used. Additional PK determinations that construct a time course of drug in anterior and posterior chamber tissues may be performed if indicated and resources are available.

Using naive animals, efficacious dosing schedules will be determined by SA1. Ocular exams, fundus imaging, and electroretinography will be performed pre-dose, Week 1 and Week 2. At termination eyes will be collected, fixed for H&E staining and reviewed by a retinal pathologist.

It is anticipated that a bioanalytical method will be established and that tissue concentrations can be measured reliably.

Potential negative results include unusually high accumulation of test article in ocular tissues which could be a precursor for toxicity in long term treatment. However, in previous studies using WBAR, such accumulation was not observed. Unexpected toxicity discovered will be examined with histopathology. In either case, lower drug concentrations or alternative formulations may be required.

### Example 5

### Rabbit Study

All reagents were obtained from Sigma-Aldrich with the exception that sesame oil was obtained from Sawall, 2965 Oakland Dr, Kalamazoo, Mich. The ethyl ester of SD-8381 was formulated in sesame oil as a saturated solution at room temperature. Saturation was assured by warming under water warm to the touch for 10 minutes and noting that upon returning to room temperature the powder was in equilibrium with what was apparently a crystalline form of the drug in that the small amount of remaining drug was now transparent (for example, the compound had clearly dissolved and crystallized out of solution). The formulation was checked for topical instillation irritancy. Two male DB rabbits were sedated with isoflurane vapors to effect. Eyes were anesthetized with topical proparacaine, the area treated with ophthalmic betadine and rinsed with sterile saline. The test article was injected (0.05 mL) as a subconjunctival DEPOT to two male DB rabbits, OU using a 29 g needle. No apparent adverse events, such as excessive scratching, redness or other signs of irritation and pain were noted.

The rabbits were sacrificed at a 6-days. Samples collected were (bilateral) aqueous humor, iris plus ciliary body, cornea, conjunctiva, sclera, choroid, retina, lens, vitreous humor and plasma which were placed in tared vials and stored at −20° C. prior to analysis. Care was taken to not cross contaminate fields by dissecting high concentration tissues as separately as possible from other tissues.

The free acid of SD-8381 was formed enzymatically using ethyl 6,8-dichloro-2-(trifluoromethyl)-2H-chromene-3-carboxylate and porcine esterase (all reagents and chemicals are from Sigma-Aldrich). The reaction was initiated by adding 5 mg of compound in methanol to 8 mg of esterase in 250 μL of 50 mM potassium phosphate pH 8 buffer. The reaction proceeded at 37° C. and was complete after 3 hours. Conversion of the methyl ester to the free acid was monitored using HPLC-UV with 254 nm detection and confirmed by flow injection analysis using a Waters Quattro Micro mass spectrometer in negative ion mode at unit resolution. SD-8381 free acid was purified by HPLC fraction collection using a 4.6×250 mm Agilent Eclipse XDB-C18 column using 0.1% formic acid/H2O/acetonitrile mobiles phases. Fractions containing the free acid were dried in a weighed tube using a SpeedVac concentrator.

Free acid SD-8381 was reconstituted in methanol to establish w/v concentration standards for LC-MS analysis. Analytical standards were prepares using EDTA mouse plasma from BioVT. A dilution series of free acid SD-8381 in methanol were spiked into mouse plasma and each concentration was combined with 3 volumes of methanol containing 1 μM diclofenac as a surrogate internal standard as no stable-isotope labeled internal standard (SIL-IS) of SD-8381 is currently available. Standards and plasma blanks were vortex mixed and centrifuged at 13K×g for 5 minutes. Supernatant was transferred to glass insert vials and immediately analyzed by LC-MS analysis. Tissue samples were homogenized using a GE Healthcare sample grinding kit #80-6483-37 and 1 volume of DPBS containing 50% methanol. Proteins were precipitated using 2 additional volumes of methanol and vortex mixing for 5 minutes. Samples were centrifuged at 13K×g for 5 minutes. Supernatant was removed and dried in a SpeedVac concentrator under medium heat. Sample was reconstituted using methanol containing 1 uM Diclofenac. Samples were analyzed using a Waters nanoAcquity UPLC in-line with a high-resolution Waters Synapt G2-S mass spectrometer via an electrospray ion source. UPLC separation of used performed using a 0.375 mm×10 cm Waters XBridge C18 3.5 u column and a flow rate of 10 μL per minute. Mobile phases were 0.1% formic acid in water as mobile phase A and 0.1% formic acid in acetonitrile as mobile phase B. Gradient elution was accomplished with 1% B initial sample loading with ramping to 95% B at 3 minutes, holding at 95% B for 5 minutes, and returning to 1% B at 8.1 minutes. Total run time was 12 minutes and 5 μL of sample was injected per analysis. Mass spectrometry data was collected over a mass range of 100-600 Da as 0.5 sec scans. Waters Masslynx 4.1 software was used for instrument control and Quanlynx application manager software was used for data analysis and exporting data to Excel.

Results Retina-choroid: The individual values for retina were 36.8, 2.01, 9.14 and 4.37 μM and for choroid were 322, 9.78, 2.43 and 50.4 μM. The high value in retina and choroid were from the same subject, same side and when these values were dropped, the average for retina was 5.17 μM and for choroid was 20.9 μM.

For comparison, the concentrations in the retina-choroid for a DEPOT of a similar lipophilic small molecule, dexamethasone, when placed very close to the retina (sub-Tenon injection) were 0.31 and 0.93 μM [Huang Z, Yang W, Zong Y, Qiu S, Chen X, Sun X, Zhou Y, Xie Z, and Gao Q. A study of the dexamethasone sodium phosphate release properties from a periocular capsular drug delivery system. Drug Delivery 2016:23; 839-847]. This difference is almost two orders of magnitude less for a compound of similar MW and lipophilicity supporting our study that SD-8381 has the necessary properties to effectively cross the sclera. Also, these dexamethasone data are for a situation where drug delivery has been optimized.

The concentrations at steady-state of SD-8381 in the retina-choroid when compared with one of the most studied NSAIDs in the literature, nepafenac (2-amino-3-benzoylbenzeneacetamide) following topical administration, was again approximately two orders of magnitude higher. The concentrations of nepafenac in the posterior (anterior) retina were 0.00272 (0.110) μM and in the posterior (anterior) choroid were 0.0551 (0.714) μM. The concentrations of amfenac (2-amino-3-benzoylbenzeneacetic acid), an active metabolite of nepafenac, in the posterior (anterior) retina were 0.000765 (0.011) μM and in the posterior (anterior) choroid were 0.0418 (0.468) μM [Chastain J E, Sanders M E, Curtis M A, Chemuturi N V, Gadd M E, Kapin M A, Markwardt K L, Dahlin D C. Distribution of topical ocular nepafenac and its active metabolite amfenac to the posterior segment of the eye. Exp Eye Res. 2016:145; 58-67]. Plasma concentrations were low as expected at 43 and 10 ng/mL, 10²-10⁴ less than ocular tissues.

The results of the above study and comparisons are illustrated below in Table No. 1.

TABLE NO. 1
Rabbit Study Results and Comparisons

| | | Dexamethasone⁴⁰ | | |
|---|---|---|---|---|
| | SD-8381 | DEPOT | Nepafenac⁴¹ | Amfenac⁴¹ |
| | DEPOT | sub-Tenon's sac | Topical | Topical |
| | *Dropped High Value | | Posterior | Posterior |
| | Same Subject | | (Anterior) | (Anterior) |
| | Time | | | |
| | Steady-State | Steady-State | Cmax | Cmax |
| | Units | | | |
| | μM | μM | μM | μM |
| Retina | 13 (n = 4) | 0.31 | 0.0027 | 0.00077 |
| | 5.2 (n = 3)* | | (0.11) | (0.011) |
| Choroid | 96 (n = 4) | 0.92 | 0.055 | 0.042 |
| | 21 (n = 3)* | | (0.71) | (0.47) |

In summary, these data support that SD-8381 has sufficient flux across the Sclera allowing for drug delivery to tissues inside the sclera including the retina and the choroid to achieve a desired therapeutic effect. The DEPOT study represented the most direct study to perform with the fewest variables. With a DEPOT study, a known amount of drug is placed outside of the eye and drug flux across the sclera to the retina-choroid can be evaluated. The μM concentrations of SD-8381 observed are only possible with significant flux across the sclera, since drug in choroid is being rapidly removed by circulation. These data support our theory that a combination of high lipophilicity, and high water solubility relative to the observed lipophilicity so as to effectively cross the hydrophilic sclera environment, results in effective drug delivery from outside the globe to the retina-choroid. From the highly concentrated prodrug DEPOT, obtaining a medium to high μM concentration near sclera is reasonable and the μM concentrations in retain-choroid can occur with effective drug delivery across the sclera. SD-8381 is highly bound to plasma proteins, thusly, removal of SD-8381 by the blood of SD-8381 in the choroid (retina) would be expected. This strongly supports the interpretation that rapid flux across the sclera to replace the rapidly removed SD-8381 in the retina-choroid was achieved inn this study.

### REFERENCES

1. Scott D. Schoenberg S D, Kim S J. Nonsteroidal Anti-Inflammatory Drugs for Retinal Disease. Int. J. Inflam., 2013:2013:1-8.
2. Wang J L, Carter J, Kiefer J R, Kurumbail R G, Pawlitz J L, Brown D, Hartmann S J, Graneto M J, Seibert K, Talley J J. The novel benzopyran class of selective cyclooxygenase-2 inhibitors-part I: the first clinical candidate. Bioorg. Med. Chem. Lett. 2010 Dec. 1; 20(23):7155-7158.
3. Wang J L, Aston K, Limburg D, Ludwig C, Hallinan A E, Koszyk F, Hamper B, Brown D, Graneto M, Talley J, Maziasz T, Masferrer J, Carter J. The novel benzopyran class of selective cyclooxygenase-2 inhibitors. Part III: the three microdose candidates. Bioorg. Med. Chem. Lett. 2010:20; 7164-7168.
4. Wang J L, Limburg D, Graneto M J, Springer J, Hamper J R, Liao S, Pawlitz J L, Kurumbail R G, Maziasz T, Talley J J, Kiefer J R, Carter J. The novel benzopyran class of selective cyclooxygenase-2 inhibitors. Part 2: The second clinical candidate having a shorter and favorable human half-life. Bioorg. Med. Chem. Lett. 2010 Dec. 1; 20(23):7159-7163.
5. Vrbanac J J, Buhl A E, Humphrey S J, Lane K E Benchmarking Topical Instillation for Ocular Therapy. SBIR Grant Submission PHS 2003-2, 2003.
6. Li Y, Burke J A, Vilupuru A S, Tsai S, Lin T, Ghosn C, Whitcup S M, Wheeler L A. Effectiveness of Dexamethasone Intravitreal Implant in an Animal Model of Chronic Retinal. Neovascularization. Invest. Ophthalmol. Vis. Sci. 2010; 51(13):5327.
7. Hu W, Brookes R, Lewis A, Woodley V, James D, Henry S, Goody R J, Attwood J, Lawrence M S, Hu W; Characterization of DL-2-aminoadipicacid-induced retinal neovascularization and leakage in nonhuman primates. Invest. Ophthalmol. Vis. Sci. 2016; 57(12):4179.
8. del Amo E A, et. al. Pharmacokinetic aspects of retinal drug delivery. Prog. in Ret. Eye Res. 2017:57:134-158.
9. Zhang R, Liu Z, Zhang H, Zhang Y, Lin D. The COX-2-Selective Antagonist (NS-398) Inhibits Choroidal Neovascularization and Subretinal Fibrosis. Plops One. 2016 Jan. 13; 11(1).
10. Scott D. Schoenberg S D, Kim S J. Nonsteroidal Anti-Inflammatory Drugs for Retinal Disease. Int. J. Inflam., 2013:2013:1-8.
11. O'Brien, William J. Therapy with 9-b-D-arabinofuranosyladenine (ARA-A) and 2′-deoxycoformycin increases the ARA-A content of ocular tissues. (Med. Coll. Wisconsin, Eye Inst., Milwaukee, Wis., 53226, USA). Curr. Eye Res., 1986:5(8), 595-599.
12. Li Y, Busoy J M, Zaman B A A, Tan Q S W, Tan G S W, Barathi V A, Cheung N, Wei J J, Hunziker W, Hong W, Wong T Y, Cheung C M G. A novel model of persistent retinal neovascularization for the development of sustained anti-VEGF therapies. Exp Eye Res. 2018 September; 174:98-106.
13. Shen W, Li S, Gillies M C; Selective Disturbance of MüLler Cell Function Leads to Retinal Vascular Changes in Rats. Invest. Ophthalmol. Vis. Sci. 2008; 49(13):3973.
14. Ayalasomayajula S P, Kompella U B. Retinal delivery of celecoxib is several-fold higher following subconjunctival administration compared to systemic administration. Pharm Res. 2004 October; 21(10):1797-804.
15. Kima S J, Tomaa H S, Barnett J M, Pennab J S., Ketorolac inhibits choroidal neovascularization by suppression of retinal VEGF. Exp. Eye. Res. 2010:91; 537-543.
16. Castro MR1, Lutz D, Edelman J L. Effect of COX inhibitors on VEGF-induced retinal vascular leakage and experimental corneal and choroidal neovascularization. Exp Eye Res. 2004 August; 79(2):275-85.
17. Mei Fl, Wang J G, Chen Z J, Yuan Z L. Effects of epoxyeicosatrienoic acids (EETs) on retinal macular degeneration in rat models. Eur Rev Med Pharmakon Sci. 2017 June; 21(12):2970-2979.
18. Shimazawa, Masamitsu; Inoue, Yuki; Masuda, Tomomi; Onodera, Risako; Tahara, Kohei; Shimizu, Yoshitaka; Mibe, Yasuhiko; Tsuruma, Kazuhiro; Takeuchi, Hirofumi; Hara, Hideaki. Topical Diclofenac-Loaded Liposomes Ameliorate Laser-Induced Choroidal Neovascularization in Mice and Non-Human Primates. Current Neurovascular Research, Volume 14, Number 1, 2017, pp. 46-52(7).
19. J J Vrbanac, T L VandeGiessen, B W Jones, L R Norris, Y Hansson, A Valmari, S Singh, Latanoprost and Timolol Pharmacokinetics in Plasma and Aqueous Humor in the Rabbit After Ocular Administration. Association for Research in Vision and Ophthalmology, Ft. Lauderdale, 4-9 May 2003.
20. J J Vrbanac, L A Adams, M R Schuette, S Yamazaki, T L VandeGiessen, B W Jones, L R Norris, M Shawer, S K Singh and T A Birkebak. Drug Pharmacokinetics in Rat Eye Tissues Following Topical Administration. Association for Research in Vision and Ophthalmology, Ft. Lauderdale, 4-9 May 2003.
21. S K Singh, M Shawer, P K Andrus, T J Raub, J J Vrbanac. Ocular Delivery Screening Scheme for Systemically Administered Ophthalmic Drugs. Association for Research in Vision and Ophthalmology, Ft. Lauderdale, 4-9 May 2003.
22. Vrbanac J J, L A Adams, M R Schuette, S Yamazaki, T L VandeGiessen, B W Jones, L R Norris, M Shawer, S K Singh and T A Birkebak. Drug Pharmacokinetics in Rat Eye Tissues Following Topical Administration. Association for Research in Vision and Ophthalmology, Ft. Lauderdale, 4-9 May 2003.
23. Acheampong, Andrew A; Shackleton, Martha; John, Brian; Burke, James; Wheeler, Larry; Tang-Liu, Diane. Distribution of brimonidine into anterior and posterior tissues of monkey, rabbit, and rat eyes. Drug Metabolism and Disposition, 2002:30(4); 421-429.
24. Acheampong, Andrew A.; Breau, Alan; Shackleton, Martha; Luo, Wendy; Lam, Steve; Tang-Liu, Diane D.-S. Comparison of concentration-time profiles of levobunolol and timolol in anterior and posterior ocular tissues of albino rabbits. J. Ocul. Pharmacol. Ther., 1995:11(4); 489-502.
25. Lipinski C A, Lombardo F, Dominy B W Feeney P J. Experimental and computational approaches to estimate solubility and permeability in drug discovery and development settings. Adv Drug Delivery Rev 1997:23; 3-25.
26. Takahashi Kyoichi; Saishin Yoshitsugu; Saishin Yumiko; Mori Keisuke; Ando Akira; Yamamoto Satoru; Oshima Yuji; Nambu Hiroyuki; Melia Michele B; Bingaman David P; Campochiaro Peter A. Topical Nepafenac inhibits ocular neovascularization. IVOS 2003:44; 409-415.
27. Maurice David M. Drug delivery to the posterior segment from drops. Survey of Ophthalmol 2002:47(Suppl 1); S41-52.
28. Geroski D H, Edelhauser H F. Drug delivery for posterior segment eye disease. IOVS 2000:41; 961-964.
29. Huang Z, Yang W, Zong Y, Qiu S, Chen X, Sun X, Zhou Y, Xie Z, and Gao Q. A study of the dexamethasone sodium phosphate release properties from a periocular capsular drug delivery system. Drug Delivery 2016:23; 839-847.
30. Chastain J E, Sanders M E, Curtis M A, Chemuturi N V, Gadd M E, Kapin M A, Markwardt K L, Dahlin D C. Distribution of topical ocular nepafenac and its active metabolite amfenac to the posterior segment of the eye. Exp Eye Res. 2016:145; 58-67.

All references, patents and patent publications are incorporated herein by reference.

The embodiments mentioned above are merely several implementations of the present disclosure. Although these embodiments have been described specifically and in details, these embodiments shall not be regarded as any limitation to the present disclosure. It should be noted that, those skilled in the art may make various variations and improvements without departing from the concept of the present invention, and those variations and improvements shall fall into the protection cope of the present disclosure.

What is claimed is:

1. A pharmaceutical composition suitable for topical administration to an eye of a subject, the composition comprises a selective cyclooxygenase-2 inhibitory drug or a pharmaceutically acceptable prodrug or salt thereof, for the treatment or prophylaxis of an ophthalmic disease or disorder in the subject, the cyclooxygenase-2 inhibitory drug having water solubility of equal to or greater than about 0.2 mg/ml, a Log P of between about 2.5 to about 5.5, and a potency of less than or equal to about 50 nM.

2. The pharmaceutical composition of claim 1, wherein the composition is in a therapeutically-effective concentration useful for the treatment and/or prophylaxis of wet age-related macular degeneration disease.

3. The pharmaceutical composition of claim 1, wherein the composition includes at least one ophthalmically acceptable excipient.

4. The pharmaceutical composition of claim 1, wherein the selective cyclooxygenase-2 inhibitory drug exhibits a Log P of greater than about 5.1.

5. The pharmaceutical composition of claim 1, wherein the selective cyclooxygenase-2 inhibitory drug exhibits a water solubility of equal to or greater than about 40 mg/ml.

6. The pharmaceutical composition of claim 1, wherein the selective cyclooxygenase-2 inhibitory drug exhibits a water solubility of equal to or greater than about 20 mg/ml.

7. The pharmaceutical composition of claim 1, wherein the selective cyclooxygenase-2 inhibitory drug exhibits a water solubility of equal to or greater than about 5 mg/ml.

8. The pharmaceutical composition of claim 1, wherein the selective cyclooxygenase-2 inhibitory drug exhibits a water solubility of equal to or greater than about 1 mg/ml.

9. The pharmaceutical composition of claim 1, wherein the selective cyclooxygenase-2 inhibitory drug exhibits a potency of less than or equal to about 25 nM.

10. The pharmaceutical composition of claim 1, wherein selective cyclooxygenase-2 inhibitory drug exhibits a potency of less than or equal to about 10 nM.

11. The pharmaceutical composition of claim 1, wherein the selective cyclooxygenase-2 inhibitory drug exhibits a resonance time in the eye equal to or greater than about 60 minutes.

12. The pharmaceutical composition of claim 11, wherein the selective cyclooxygenase-2 inhibitory drug exhibits a resonance time in the eye of less than or equal to about 36 hours.

13. The pharmaceutical composition of claim 1, wherein the pharmaceutical composition is formulated for topical instillation or a periocular DEPOT injection to the eye.

14. The pharmaceutical composition of claim 1, wherein the selective cyclooxygenase-2 inhibitory drug is ethyl 6,8-dichloro-2-(trifluoromethyl)-2H-chromene-3-carboxylate, or a pharmaceutically acceptable prodrug or salt thereof.

15. A method of treating a cyclooxygenase-2 mediated ophthalmic disease or disorder in a subject having or susceptible to the ophthalmic disease or disorder, the method comprising applying to an eye the subject a therapeutically-effective amount of a selective cyclooxygenase-2 inhibitory drug having a Log P between about 2.5 to about 5.5.

16. The method of claim 15, wherein the ophthalmic disease or disorder is wet age-related macular degeneration.

17. The method of claim 15, wherein the selective cyclooxygenase-2 inhibitory drug is applied via a topical instillation or a periocular DEPOT injection.

18. A kit comprising at least one selective cyclooxygenase-2 inhibitory drug, or a pharmaceutically acceptable prodrug or salt thereof, at least one ophthalmically acceptable ingredient, and a device for administering the at least one selective cyclooxygenase-2 inhibitory, or the pharmaceutically acceptable prodrug or salt thereof, and the at least one ophthalmically acceptable ingredient to the eye of the subject.

19. The kit of claim 18, where the subject has or is susceptible to a wet age-related macular degeneration disease.

20. The kit of claim 18, wherein the administration comprises a topical instillation or a periocular DEPOT injection.
